(19) United States
(12) Patent Application Publication
(10) Pub. No.: US 2025/0177709 A1
(43) Pub. Date: Jun. 5, 2025

(54) VAGINAL APPLICATOR

(71) Applicant: Azadeh RAZZAGH SHOAR, Toronto (CA)

(72) Inventor: Azadeh RAZZAGH SHOAR, Toronto (CA)

(21) Appl. No.: 18/952,230

(22) Filed: Nov. 19, 2024

Related U.S. Application Data
(60) Provisional application No. 63/606,238, filed on Dec. 5, 2023.

(51) Int. Cl. A61M 31/00

(52) U.S. Cl. CPC A61M 31/00; A61M 2210/1475

(57) ABSTRACT

A vaginal applicator that has an applicator body and a plunger. The applicator body is formed as an elongate arcuated hollow cylinder defined by a sidewall and having a bore diameter. The applicator body has a rounded closed end and an open end, and has a plurality of holes defined in its rounded closed end and at least a portion of the sidewall proximate the rounded closed end. The plunger has an elongate arcuated shaft and a rounded end, wherein the rounded end has a diameter matching the bore diameter.

### CROSS-REFERENCE TO RELATED APPLICATIONS

The present application claims priority to U.S. patent application Ser. No. 63/606,238 filed Dec. 5, 2023, the contents of which are hereby incorporated by reference.

### FIELD

The present disclosure relates to vaginal applicators for delivering creams, gels, or other substances intravaginally.

### BACKGROUND

Vaginal applicators may be used for a variety of purposes. In many cases, they are designed to deliver a suppository or a medicated cream or gel within the vagina with the intention of applying the cream or gel to the membranes of the vagina or the cervix. For example, a vaginal applicator may be used to administer medicated creams or gels in the treatment of vaginal infections or inflammations. Bacterial Vaginosis and yeast infections are common vaginal conditions in women that can be treated through the intravaginal administration of a medicated cream or gel. However, common vaginal applicators on the market for administering medication intravaginally may cause discomfort and even pain for some users due to poor design.

### BRIEF DESCRIPTION OF THE DRAWINGS

Reference will now be made, by way of example only, to the accompanying drawings which show example embodiments of the present application, in which:

FIG. 1 shows a perspective view of one example of a vaginal applicator in disassembled form.

FIG. 2 shows a perspective view of the vaginal applicator in an assembled form.

FIG. 3 shows a perspective view with a plunger partly inserted in an applicator body of the vaginal applicator.

FIG. 4 shows a side view of the vaginal applicator.

FIG. 5 shows a close-up perspective view of a portion of the vaginal applicator.

FIG. 6 shows a close-up view of a rounded closed end of the vaginal applicator.

FIG. 7 shows an end-on perspective view of the vaginal applicator.

### DETAILED DESCRIPTION

In one aspect, the present application describes a vaginal applicator. The vaginal applicator may include an applicator body formed as an elongate arcuated hollow cylinder defined by a sidewall and having a bore diameter, the applicator body having a rounded closed end and an open end, the applicator body having a plurality of holes defined in its rounded closed end and at least a portion of the sidewall proximate the rounded closed end; and a plunger having an elongate arcuated shaft and a rounded end, wherein the rounded end has a diameter matching the bore diameter.

In some implementations, the applicator body may further include an outwardly extending flange around its open end to form a handle. The plunger may include a plunger flange opposite the rounded end. In some cases, the outwardly extending flange forms a concave face and the plunger flange nests in the concave face when the plunger is fully inserted in the applicator body.

In some implementations, the rounded end includes a cylindrical portion and a curved end portion. In some cases, the elongate arcuated shaft is smaller in diameter than the bore diameter. In some cases, the cylindrical portion has a diameter matching the bore diameter and the curved end portion nests within the rounded end of the applicator body when the plunger is fully inserted in the applicator body.

In some implementations, the applicator body has an arcuate curve and wherein the elongate arcuate shaft has a curve matching the arcuate curve.

In some implementations, the applicator body is formed from silicon.

In some implementations, the applicator body is formed from one of polyethylene terephthalate, high-density polyethylene, or polypropylene.

In another aspect, the present application describes a medicinal kit. The kit may include a vaginal applicator and a medicated cream for insertion into the vaginal applicator. The applicator may include an applicator body formed as an elongate arcuated hollow cylinder defined by a sidewall and having a bore diameter, the applicator body having a rounded closed end and an open end, the applicator body having a plurality of holes defined in its rounded closed end and at least a portion of the sidewall proximate the rounded closed end; and a plunger having an elongate arcuated shaft and a rounded end, wherein the rounded end has a diameter matching the bore diameter.

In yet a further aspect, the present application describes a vaginal applicator. The applicator may include an elongate curved applicator body formed using a hollow cylinder having a rounded closed end and an open end, the applicator body having a plurality of holes defined in its rounded closed end and at least a portion of its sidewall proximate the rounded closed end; and a plunger having an elongate arcuated shaft and a rounded end, the plunger having an arcuate curve that matches an arcuate curve of the applicator body.

In some implementations, the applicator body further includes an outwardly extending flange around its open end to form a handle. The plunger may include a plunger flange opposite the rounded end. The outwardly extending flange may form a concave face and the plunger flange may nest in the concave face when the plunger is fully inserted in the applicator body.

In some implementations, the rounded end includes a cylindrical portion and a curved end portion. The elongate arcuated shaft may be smaller in diameter than an interior diameter of the hollow cylinder. The cylindrical portion may have a diameter matching the interior diameter and the curved end portion nests within the rounded end of the applicator body when the plunger is fully inserted in the applicator body.

In some implementations, the rounded end is formed from silicon.

In some implementations, the applicator body is formed from one of silicon, polyethylene terephthalate, high-density polyethylene, or polypropylene.

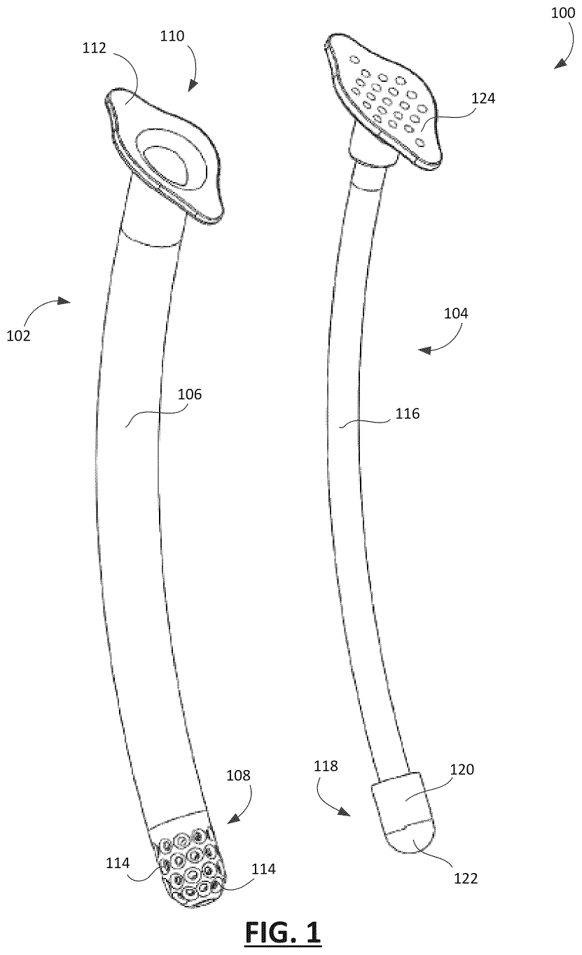

Reference is first made to FIG. 1, which shows a perspective view of one example of a vaginal applicator 100 in disassembled form, in accordance with an aspect of the present application. The vaginal applicator 100 includes an applicator body 102 and a plunger 104. The applicator body 102 is formed from an elongate arcuated hollow cylinder 106. The hollow cylinder 106 is formed from a sidewall and it has a bore diameter defining the width or size of its hollow interior. The applicator body 102 has an open end 110 and a rounded closed end 108.

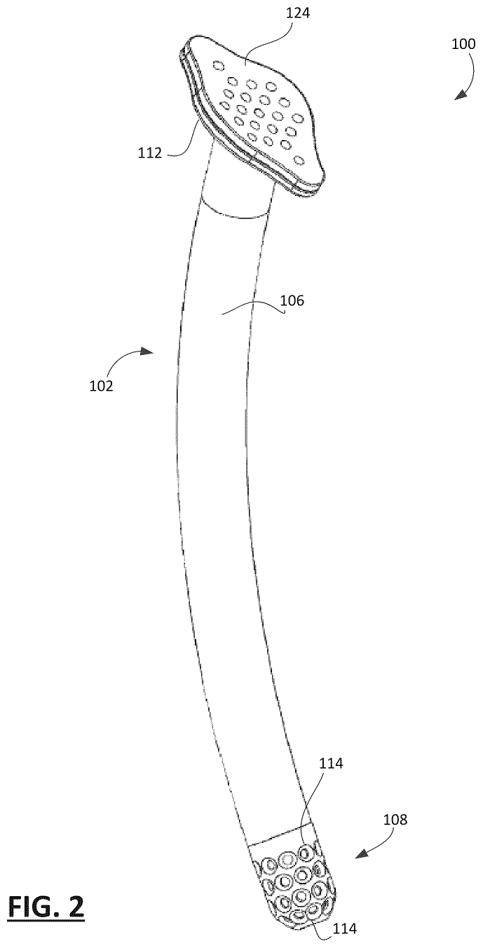

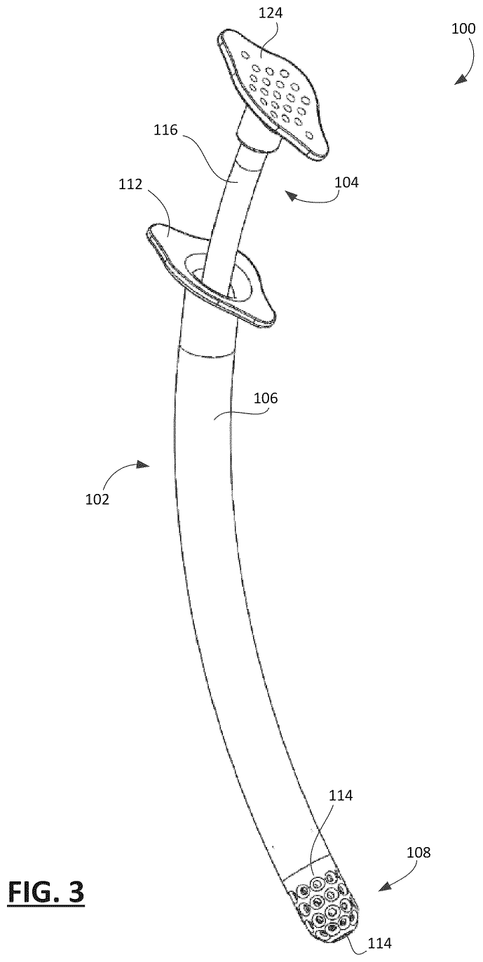

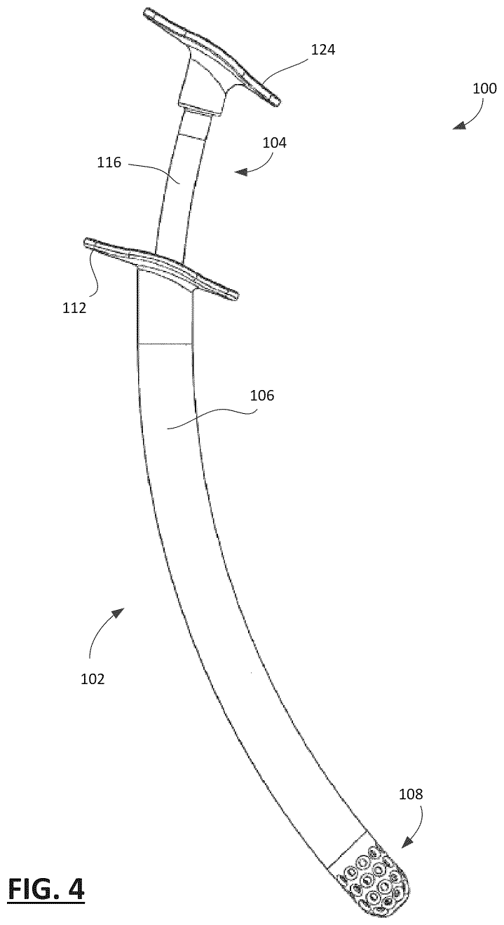

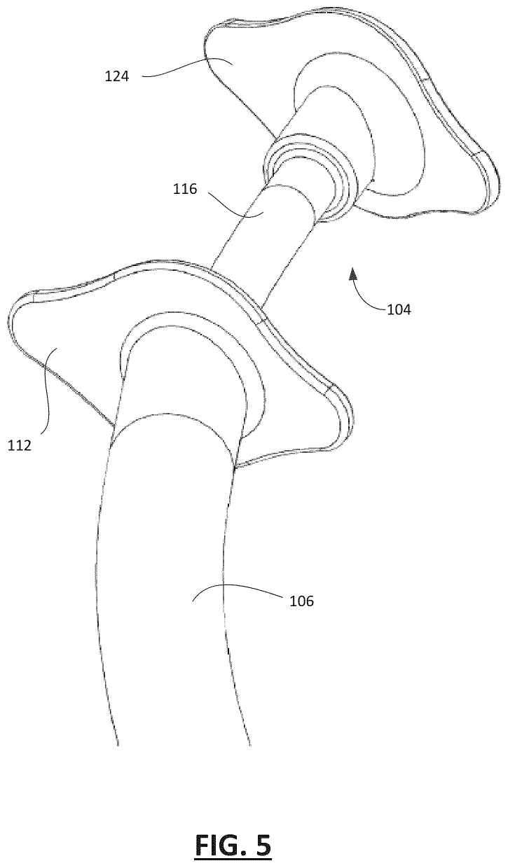

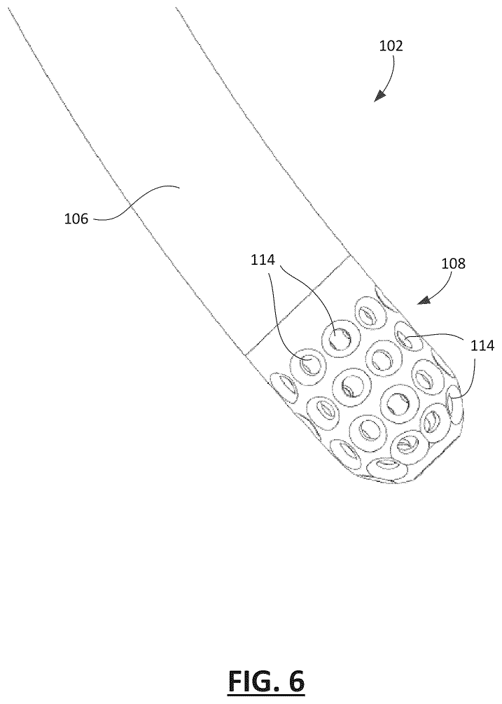

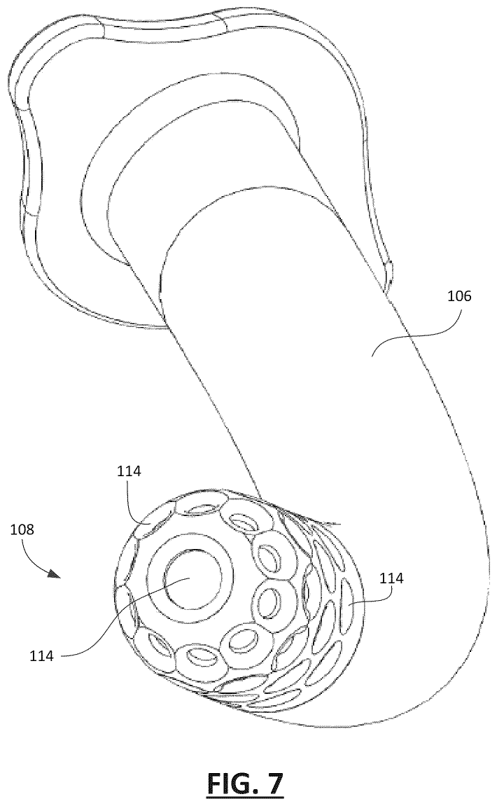

Reference is also made to views of the vaginal applicator 100 shows in FIGS. 2 through 7. FIG. 2 shows a perspective view of the vaginal applicator 100 in an assembled form with the plunger 104 fully inserted in the applicator body 102. FIG. 3 shows a perspective view with the plunger 104 partly inserted in the applicator body 102. FIG. 4 shows a side view with the plunger 104 partly inserted in the applicator body 102. FIG. 5 shows a close-up perspective view of the plunger 104 partly inserted in the applicator body 102. FIG. 6 shows a close-up view of the rounded closed end 108 of the applicator body 102. FIG. 7 shows an end-on perspective view of the vaginal applicator 100.

It will be appreciated that both the applicator body 102 and the plunger 104 are curved. Through use of the elongate arcuated hollow cylinder 106 the applicator body 102 features a gentle curvature in its shape that is designed to better accommodate insertion in the vagina by a user. The curved shape fits better anatomically and enables a more comfortable angle of insertion.

The plunger 104 includes an elongate arcuated shaft 116 having the same angle of curvature as the applicator body 102, thereby enabling the plunger 104 to be inserted into the elongate arcuated hollow cylinder 106. That is, the applicator body 102 is shaped to have an arcuate curve and the elongate arcuated shaft 116 has a curve matching the arcuate curve.

The plunger 104 further includes a rounded end 118. In some implementations, the rounded end 118 may be formed from a cylindrical portion 120 and a curved end portion 122. The cylindrical portion 120 may be sized to have a diameter that matches the bore diameter such that when the plunger 104 is inserted into the applicator body 102 the rounded end 118 touches the inner sidewall of the hollow cylinder 106. In some implementations, the rounded end 118 may be formed from a plastic or from silicone. The rounded end 118 may be slightly malleable such that it can be slightly deformed under the pressure of the surrounding sidewall to shape to it. The rounded end 118 may bear against the surface of the inner sidewall while the plunger 104 is inserted or withdrawn from the applicator body 102. The curved end portion 122 may be shaped and sized to match the inner wall shape and size of the rounded closed end 108 of the applicator body 102.

The applicator body 102 features a plurality of holes 114 creating passages through the sidewall and, in particular, in the rounded closed end 108 and, in some cases, in a portion of the hollow cylinder 106 sidewall proximate the rounded closed end 108. The plurality of holes 114 ensure that any air and other substances within the applicator body 102, such as medicated creams or gels, are extruded through the holes 114 as the plunger 104 is inserted into the applicator body 102. During insertion, the rounded end 118 pushes the cream or gel within the hollow cylinder 106 towards the rounded closed end 108, where it is ejected from the applicator body 102 through the plurality of holes 114, thereby distributing the cream or gel around the exterior surface of the rounded closed end 108 and the end of the hollow cylinder 106 proximate the rounded closed end 108. In some implementations, the plurality of holes 114 are formed in the portion of the hollow cylinder 106 that will be in contact with the cylindrical portion 120 of the rounded end 118 when the plunger 104 is fully inserted.

Some vaginal applicators feature a large open end or a single central aperture in a closed end, which results in the applicator ejecting all cream or gel in a jet in an axial direction. This may result in misapplication of the cream or gel. By extruding the cream or gel from multiple holes spaced around the rounded closed end 108 and along a portion of the hollow cylinder 106, the vaginal applicator 100 ensures that the cream or gel is brought into contact with many points of the vaginal membrane. Moreover, by extruding the cream or gel from holes in the sidewall, the vaginal applicator 100 is more likely to rub or smear the cream or gel broadly on the vaginal membranes, ensuring that any infected or inflamed areas are treated directly. As a result, the vaginal applicator 100 may be expected to have greater efficacy in treating intravaginal conditions than other applicators.

Although the plurality of holes 114 are shown as being cylindrical, in some implementations they may be other shapes. The plurality of holes 114 may all be the same size or may be different sizes. In one example, as shown in FIG. 7, the hole 114 at the very tip of the rounded closed end 108 may be larger than the other holes 114 in the rounded closed end 108.

The applicator body 102 may include an outwardly extending flange 112 at its open end. The plunger 104 may include a corresponding plunger flange 124. The outwardly extending flange 112 may have a slightly concave face and the plunger flange 124 may have a corresponding shape such that it nests within the concave shape of the outwardly extending flange 112 when plunger 104 is fully inserted in the applicator body 102. The slightly concave shaping of the flanges 112, 124 makes them ergonomically easy to handle and actuate.

In some implementations, the elongate arcuated shaft 116 of the plunger 104 may be cylindrical as shown in the example embodiment. However, the elongate arcuated shaft 116 may have other cross-sectional shapes in other embodiments. In one example, the elongate arcuated shaft 116 may have a cross-sectional shape of a plus sign or cross to reduce the amount of material used in forming the plunger 104 but to give it sufficient rigidity. Other shapes may be used in other implementations.

In some cases, the plunger 104 may include one or more spacers (not illustrated) around the elongate arcuated shaft 116. The spacers may be shaped as thin transverse disks having a diameter that matches the bore diameter of the hollow cylinder 106.

In some implementations, the applicator body 102 may be formed from silicone. In some implementations, the applicator body 102 may be formed from a plastic. The plunger 104 may also be formed from a plastic in some cases. Example plastics that may be used in some cases include polyethylene terephthalate, high-density polyethylene, or polypropylene. In some cases, one or both of the applicator body 102 and the plunger 104 may be made from a combination of materials.

As noted, certain adaptations and modifications of the described embodiments can be made. Therefore, the above discussed embodiments are to be considered illustrative and not restrictive.

1. A vaginal applicator, comprising:
an applicator body formed as an elongate arcuated hollow cylinder defined by a sidewall and having a bore diameter, the applicator body having a rounded closed end and an open end, the applicator body having a plurality of holes defined in its rounded closed end and at least a portion of the sidewall proximate the rounded closed end; and
a plunger having an elongate arcuated shaft and a rounded end, wherein the rounded end has a diameter matching the bore diameter.

2. The vaginal applicator of claim 1, wherein the applicator body further includes an outwardly extending flange around its open end to form a handle.

3. The vaginal applicator of claim 2, wherein the plunger includes a plunger flange opposite the rounded end.

4. The vaginal applicator of claim 3, wherein the outwardly extending flange forms a concave face and wherein the plunger flange nests in the concave face when the plunger is fully inserted in the applicator body.

5. The vaginal applicator of claim 1, wherein the rounded end includes a cylindrical portion and a curved end portion.

6. The vaginal applicator of claim 5, wherein the elongate arcuated shaft is smaller in diameter than the bore diameter.

7. The vaginal applicator of claim 6, wherein the cylindrical portion has a diameter matching the bore diameter and the curved end portion nests within the rounded end of the applicator body when the plunger is fully inserted in the applicator body.

8. The vaginal applicator of claim 1, wherein the applicator body has an arcuate curve and wherein the elongate arcuate shaft has a curve matching the arcuate curve.

9. The vaginal applicator of claim 1, wherein the applicator body is formed from silicon.

10. The vaginal applicator of claim 1, wherein the applicator body is formed from one of polyethylene terephthalate, high-density polyethylene, or polypropylene.

11. A medicinal kit, comprising:
a vaginal applicator, including
an applicator body formed as an elongate arcuated hollow cylinder defined by a sidewall and having a bore diameter, the applicator body having a rounded closed end and an open end, the applicator body having a plurality of holes defined in its rounded closed end and at least a portion of the sidewall proximate the rounded closed end; and
a plunger having an elongate arcuated shaft and a rounded end, wherein the rounded end has a diameter matching the bore diameter; and
a medicated cream for insertion into the applicator body.

12. A vaginal applicator, comprising:
an elongate curved applicator body formed using a hollow cylinder having a rounded closed end and an open end, the applicator body having a plurality of holes defined in its rounded closed end and at least a portion of its sidewall proximate the rounded closed end; and
a plunger having an elongate arcuated shaft and a rounded end, the plunger having an arcuate curve that matches an arcuate curve of the applicator body.

13. The vaginal applicator of claim 12, wherein the applicator body further includes an outwardly extending flange around its open end to form a handle.

14. The vaginal applicator of claim 13, wherein the plunger includes a plunger flange opposite the rounded end.

15. The vaginal applicator of claim 14, wherein the outwardly extending flange forms a concave face and wherein the plunger flange nests in the concave face when the plunger is fully inserted in the applicator body.

16. The vaginal applicator of claim 12, wherein the rounded end includes a cylindrical portion and a curved end portion.

17. The vaginal applicator of claim 16, wherein the elongate arcuated shaft is smaller in diameter than an interior diameter of the hollow cylinder.

18. The vaginal applicator of claim 17, wherein the cylindrical portion has a diameter matching the interior diameter and the curved end portion nests within the rounded end of the applicator body when the plunger is fully inserted in the applicator body.

19. The vaginal applicator of claim 12, wherein the rounded end is formed from silicon.

20. The vaginal applicator of claim 12, wherein the applicator body is formed from one of silicon, polyethylene terephthalate, high-density polyethylene, or polypropylene.
